(19) United States
(12) Patent Application Publication
(10) Pub. No.: US 2014/0155834 A1
(43) Pub. Date: Jun. 5, 2014

(54) Locking Interface, Cartridge Holder and Drug Delivery Device Comprising a Locking Interface

(71) Applicants: Michael Harms, Frankfurt am Main (DE); Tobias Stever, Frankfurt am Main (DE); Christiane Schneider, Frankfurt am Main (DE); Meinolf Werner, Worms (DE); Olaf Zeckai, Weinheim (DE)

(72) Inventors: Michael Harms, Frankfurt am Main (DE); Tobias Stever, Frankfurt am Main (DE); Christiane Schneider, Frankfurt am Main (DE); Meinolf Werner, Worms (DE); Olaf Zeckai, Weinheim (DE)

(21) Appl. No.: 14/232,818

(22) PCT Filed: Jul. 12, 2012
(86) PCT No.: PCT/EP2012/063638

§ 371 Date: Jan. 14, 2014

(30) Foreign Application Priority Data
Jul. 15, 2011 (EP) 11174147.6

(51) Int. Cl. A61M 5/24

(52) U.S. Cl. CPC A61M 5/24
USPC 604/187; 403/322.1

(57) ABSTRACT

A locking interface for locking a cartridge holder onto a main body of a drug delivery device comprises a locking member for locking the cartridge holder onto the main body, a user interface operable by a user, a support member, on which the locking member and the user interface are located, wherein the locking interface is configured such that a force needed for unlocking the cartridge holder and the main body is reducible by operating the user interface.

### CROSS REFERENCE TO RELATED APPLICATIONS

The present application is a U.S. National Phase Application pursuant to 35 U.S.C. §371 of International Application No. PCT/EP2012/063638 filed Jul. 12, 2012, which claims priority to European Patent Application No. 11174147.6 filed Jul. 15, 2011. The entire disclosure contents of these applications are herewith incorporated by reference into the present application.

### FIELD OF INVENTION

Locking interface, cartridge holder and drug delivery device comprising a locking interface

### BACKGROUND

The present disclosure relates to a locking interface for locking a cartridge holder onto a main body of a drug delivery device, a cartridge holder comprising such a locking interface and a drug delivery device comprising such a locking interface.

A drug delivery device may comprise a cartridge containing a drug. The cartridge may be held by a cartridge holder being attached to a main part of the drug delivery device. In a reusable device, for enabling a replacement of the cartridge, the cartridge holder is configured to be detached from the main part. In order to avoid an inadvertent detachment of the cartridge holder from the main part of the device a locking mechanism may be provided.

A reusable drug delivery device, in particular a pen-type injection device, is described in documents WO 2009/132777 A1 and WO 2009/132781 A1, for example.

### SUMMARY

It is an object of the present invention to provide a locking interface, a cartridge holder and a drug delivery device, wherein a locking function is improved.

According to one aspect of the disclosure, a locking interface for locking a cartridge holder onto a main body of a drug delivery device is provided. The locking interface comprises a locking member for locking the cartridge holder onto the main body, a user interface operable by a user and a support member, on which the locking member and the user interface are located. The locking interface is configured such that a force needed for unlocking the cartridge holder from the main body is reducible by operating the user interface.

Preferably, the locking interface is used in a reusable drug delivery device, where a replacement of a cartridge is enabled such that the device can be reused with a new cartridge. The drug delivery device may be an injection device, in particular a pen-type injection device.

Preferably, the locking interface is configured such that a locking can be achieved by applying a low force. Thereby, the risk that the cartridge holder is not completely locked onto the main body and detaches during the use of the device can be reduced. Moreover, the locking interface is preferably configured such that an unlocking of the device requires a high force, unless the user interface is operated. In this case, an inadvertent unlocking of the device can be prevented.

In a preferred embodiment, the locking interface is configured such that locking and unlocking is achieved by applying a torque on the cartridge holder or the main body of the device. Preferably, the locking interface is configured such that a torque needed for unlocking the cartridge holder and the main body is reducible by operating the user interface.

As an example, the locking interface may be located on a cartridge holder or a main part of a drug delivery device. In particular, the locking interface may be a part of the housing of the cartridge holder or the main part.

The locking member may be configured for an interaction with a mating locking member, whereby an inadvertent detachment of the cartridge holder from the main body of the drug delivery device may be prevented. In particular, the locking member may be configured for mechanically blocking a detachment movement of the cartridge holder by mechanical interaction with the mating locking member. As an example, when the locking interface comprising the locking member is located on the cartridge holder, a mating locking member may be located on the main part of the device, where the cartridge holder is attached to. In particular, the mating locking member may be located on an inner surface of the housing of the main part of the device.

The mating locking member may comprise a protrusion. In order to establish a locking of the cartridge holder onto the main part, the mating locking member may have to be pushed over the locking member. For unlocking, the mating locking member may have to be pushed in the opposite direction over the locking member. Alternatively, the mating locking member may comprise a recess, in which the locking member engages. In order to establish a locking of the cartridge holder, the locking member may have to be pushed into the recess. Preferably, the support member is resiliently biased such that the locking member is urged into the recess by the biasing force when the locking member is located over the recess.

Preferably, the locking member is configured such that an interaction with a mating locking member occurs at the end of an attachment operation. Preferably, an attachment operation of a cartridge holder onto a main part of a drug delivery device comprises a rotational movement of the cartridge holder relative to the main part. As an example, a cartridge holder may be attachable to a main part of a drug delivery device by a guide pin being guided in a guide groove. The guide pin may be located on the cartridge holder and the guide groove may be located on the main part of the device or vice versa. The guide groove may comprise an annular section or a helical section such that when the guide pin is guided in this section a rotational movement of the cartridge holder relative to the main part occurs. Preferably, the locking member is located such that an interaction with a mating locking member occurs at the end of the rotational movement. As a further example, a cartridge holder may be attachable to a main part of a drug delivery device by a screw thread. The locking member may be located such that an interaction occurs at the end of the screwing operation.

The locking interface may comprise a base, wherein the support member is resiliently connected to the base.

As examples, the base may be the housing or a part of the housing of the cartridge holder or of the main part of the drug delivery device. In particular, the base may be a part of a cylindrical housing of a pen-type device.

By the resilient connection to the base, a resilient deflection movement of the support member relative to the base may be enabled. Preferably, the locking interface is configured such that a resilient deflection occurs during a locking operation and during operation of the user interface. A resilient deflection may lead to a reduced force required for locking or unlocking the cartridge holder from the main body.

Preferably, a deflection of the support member comprises a deflection of the locking member. On a deflection, the height of the locking member relative to the base may decrease such that a force needed for pushing a mating locking member over the locking member may be reduced.

Preferably, the locking interface is configured such that when a cartridge holder is locked to the main part, the support member already deflects when a low force is exerted on the locking member. In this case, a low force is needed for pushing a mating locking member over the locking member and, thereby, for locking the device. Preferably, the locking interface is configured such that for unlocking a cartridge holder, a large force is needed to cause a deflection of the locking member by an interaction with a mating locking member. Accordingly, a large force is needed for pushing the mating locking member over the locking member and, thereby, unlocking the device.

As an example, the locking member may have the shape of a protruding lug. In this case, a mating locking member may have the shape of a mating lug. The locking member may have a slanted interaction side for facilitating the locking operation. In particular, the slanted interaction side may be on a first interaction side of the locking member, where the mating locking member has to be pushed over when the locking is established. On pushing the mating locking member over the locking member an inward deflection of the support member may occur such that the height of the locking member relative to the base decreases. The locking member may have a second interaction side which is steeper than the first interaction side. This second interaction side may have to be overcome in order to unlock the cartridge holder from the device. When the mating locking member is pushed onto the second interaction side, a higher force is required for causing an inward deflection of the support member. Thereby, the force required for locking a cartridge holder onto a main part of the device may be less than the force required for unlocking the cartridge holder from the main part.

Alternatively, a mating locking member may have the shape of a recess. When the locking member is positioned over the recess, it may be pushed into the recess by the biasing force of the support member. For unlocking the device, the support member may have to be deflected inwardly such the locking member is disengaged from the recess and a detachment movement is enabled.

The user interface may be configured such that by operating the user interface the support member deflects relative to the base.

Thereby, the locking member may deflect such that the height of the locking member decreases and a force needed for pushing a mating locking member over the locking member is reduced. In this way, by depressing the user interface, a force needed for unlocking a cartridge holder from a main part of a drug delivery device may be reduced.

Preferably, the user interface is configured such that it can be operated by a fingertip and, in particular, by the tip of a thumb. Preferably, operating the user interface means depressing the user interface.

The user interface may comprise at least one tactile member. As examples, the tactile member may comprise at least one of a rib, a knob or a rippled surface.

The tactile member may facilitate an operation of the user interface. In particular, a slipping off of the tactile member may be prevented. Moreover, the tactile member may provide a tactile and visual marking of the user interface.

In one embodiment, the user interface and the locking member are located such on the support member that, when the user interface is operated, the locking member and the user interface deflect in the same amount relative to the support member.

In this case, the user has direct control over the amount of deflection of the locking member and, thereby, over the force reduction when unlocking a cartridge holder from a main part.

The resilient connection of the support member with the base may be facilitated by at least one cut-out in the base.

The cut-out may partially encircle the support member. In particular, the cut-out may encircle the support member except from an interface region of the support member and the base. The interface region may provide the resilient connection of the support member and the base. In particular, the support member may be an integral part of the base, partially cut out from the base. Accordingly, the surface area of the support member may be partially confined by the cut-out in the base.

In one embodiment, the base may comprise several cut-outs facilitating the resilient connection. By the shape of the one or more cut-outs and, accordingly, the interface region, the resilience of the connection may be adjusted.

The support member may be resiliently connected to the base in a hinge region.

Preferably, the support member is disconnected from the base except from the hinge region. Accordingly, the support member may be encircled by a cut-out except from the hinge region. In this case, the resilient connection of the support member to the base may be established by providing the base with a cut-out along a continuous line. As an example, the hinge region may be formed by a straight interface region between the support member and the base.

As an example, the support member may have a rectangular or an approximately rectangular surface area. At one of its side faces it may be mechanically connected to the base in the hinge region and at its three remaining side faces it may be disconnected from the base by the cut-outs.

As an example, the support member may be configured in a button-like shape, which is resiliently connected to the base in the interface region and otherwise disconnected from the base by the cut-out.

In one embodiment, the base of the locking interface extends along a longitudinal axis. The support member and the user interface may be located in an offset along the longitudinal axis. Preferably, the support member and the user interface are located at the same angular position relative to the longitudinal axis.

As an example, the base may be configured as a cartridge holder. In this case, the user interface may be located in an offset relative to the locking member towards a distal end of the cartridge holder, i.e., in the direction, where a dispense interface of the device such as a needle interface is located.

In one embodiment, a locking interface for locking a cartridge holder onto a main body of a drug delivery device comprises a locking member for locking the cartridge holder onto the main body, a support member whereupon the locking member is located and a base. The support member is resiliently connected to the base, wherein the resilient connection is facilitated by at least one cut-out in the base.

According to a further aspect of the disclosure, a cartridge holder attachable to a main body of a drug delivery device is disclosed. The cartridge holder comprises a locking interface for locking the cartridge holder onto a main body of a drug delivery device. The locking interface may comprise any structural and functional features described above.

The cartridge holder may have a longitudinal axis. In the case that the locking interface comprises a hinge region, the hinge region may be oriented parallel to the longitudinal axis of the cartridge holder.

At a distal end, the cartridge holder may comprise a dispense interface. As an example, in an injection device, the cartridge holder may comprise an interface for attaching a needle. The interface may be configured as a thread such that a needle can be threadedly engaged to the cartridge holder.

In a preferred embodiment, the cartridge holder is attachable to a main body of a drug delivery device by a revolving movement, in particular by a revolving movement around an axis of rotation. As an example, the cartridge holder may be attachable to the main body by screwing the cartridge holder onto the main body. Preferably, in this case, the locking interface comprises a hinge region, wherein the hinge region extends parallel to the axis of rotation. Thereby, a tilting of the support member can be easily caused by exerting a force on the locking member in the direction of the screwing movement, i.e., in a direction perpendicular to the hinge.

The cartridge holder may comprise an interface for attaching the cartridge holder onto the main body of a drug delivery device. As an example, the interface may comprise a screw thread enabling a threaded engagement of the cartridge holder and the main body. As a further example, the interface may comprise a guide pin guidable in a guide groove of the main body of a drug delivery device.

Preferably, the locking interface is positioned near the end of the cartridge holder where the cartridge holder is attached to a main body of the device. As an example, the locking interface may be located near the end of a screw thread for screwing the cartridge holder onto the main body.

Preferably, the user interface of the locking interface is located such that it is accessible to a user when the cartridge holder is attached to a main body of a drug delivery device. Thereby, on operating the user interface, a force for unlocking the cartridge holder from the main body may be reduced. Preferably, the locking member is located such that it is not directly accessible when the cartridge holder is locked onto the main body.

According to a further aspect of the disclosure, a drug delivery device comprising a cartridge holder, a main body and a locking interface is provided. The cartridge holder is attachable to the main body and lockable to the main body by the locking interface.

The locking interface may comprise any structural and functional features as described above. The locking interface may be located on a cartridge holder as described above. In a different embodiment, the locking interface may be located on the main body of the device.

The drug delivery device may be an injection device, in particular a pen-type injection device. Preferably, the drug delivery device is a reusable device. For this aim, the cartridge holder may be configured to be detachable from the main body of the device. The main body may comprise a dose button for activating dispensing of a dose of a drug.

The cartridge holder may be attachable to the main body by a threaded engagement. The locking interface may prevent an inadvertent detachment of the cartridge holder, in particular an inadvertent unscrewing movement.

In the case that the locking interface comprises a user interface, the user interface is preferably accessible to a user when the cartridge holder is locked to the main body. Preferably, by operating the user interface a force needed for unlocking the device is reduced. In particular, a torque needed for effecting a revolving movement of the cartridge holder or the main part of the device may be reduced.

In a preferred embodiment, the drug delivery device is configured such that the cartridge holder is attachable to the main body by a revolving movement, for example by a threaded engagement. Preferably, the cartridge holder is lockable to the main body by the locking member such that the cartridge holder remains in an attached position. The locking interface may be configured such that a relatively low torque is required for locking the cartridge holder and a high torque is required for unlocking the cartridge holder. Preferably, a torque needed for unlocking the cartridge holder is reducible by operating the user interface.

The term “drug”, as used herein, means a pharmaceutical formulation containing at least one pharmaceutically active compound,

wherein in one embodiment the pharmaceutically active compound has a molecular weight up to 1500 Da and/or is a peptide, a proteine, a polysaccharide, a vaccine, a DNA, a RNA, an enzyme, an antibody or a fragment thereof, a hormone or an oligonucleotide, or a mixture of the above-mentioned pharmaceutically active compound,

wherein in a further embodiment the pharmaceutically active compound is useful for the treatment and/or prophylaxis of diabetes mellitus or complications associated with diabetes mellitus such as diabetic retinopathy, thromboembolism disorders such as deep vein or pulmonary thromboembolism, acute coronary syndrome (ACS), angina, myocardial infarction, cancer, macular degeneration, inflammation, hay fever, atherosclerosis and/or rheumatoid arthritis,

wherein in a further embodiment the pharmaceutically active compound comprises at least one peptide for the treatment and/or prophylaxis of diabetes mellitus or complications associated with diabetes mellitus such as diabetic retinopathy,

wherein in a further embodiment the pharmaceutically active compound comprises at least one human insulin or a human insulin analogue or derivative, glucagon-like peptide (GLP-1) or an analogue or derivative thereof, or exedin-3 or exedin-4 or an analogue or derivative of exedin-3 or exedin-4.

Insulin analogues are for example Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Insulin derivates are for example B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-Y-glutamyl)-des(B30) human insulin; B29-N-(N-lithocholyl-Y-glutamyl)-des(B30) human insulin; B29-N-(ω-carboxyheptadecanoyl)-des(B30) human insulin and B29-N-(ω-carboxyheptadecanoyl) human insulin.

Exendin-4 for example means Exendin-4(1-39), a peptide of the sequence H-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser-NH2.

Exendin-4 derivatives are for example selected from the following list of compounds:

H-(Lys)4-des Pro36, des Pro37 Exendin-4(1-39)-NH2,

H-(Lys)5-des Pro36, des Pro37 Exendin-4(1-39)-NH2,

des Pro36 [Asp28] Exendin-4(1-39),

des Pro36 [IsoAsp28] Exendin-4(1-39),

des Pro36 [Met(O)14, Asp28] Exendin-4(1-39),

des Pro36 [Met(O)14, IsoAsp28] Exendin-4(1-39),

des Pro36 [Trp(O2)25, Asp28] Exendin-4(1-39),

des Pro36 [Trp(O2)25, IsoAsp28] Exendin-4(1-39),

des Pro36 [Met(O)14 Trp(O2)25, Asp28] Exendin-4(1-39),

des Pro36 [Met(O)14 Trp(O2)25, IsoAsp28] Exendin-4(1-39); or

des Pro36 [Asp28] Exendin-4(1-39),

des Pro36 [IsoAsp28] Exendin-4(1-39),

des Pro36 [Met(O)14, Asp28] Exendin-4(1-39),

des Pro36 [Met(O)14, IsoAsp28] Exendin-4(1-39),

des Pro36 [Trp(O2)25, Asp28] Exendin-4(1-39),

des Pro36 [Trp(O2)25, IsoAsp28] Exendin-4(1-39),

des Pro36 [Met(O)14 Trp(O2)25, Asp28] Exendin-4(1-39),

des Pro36 [Met(O)14 Trp(O2)25, IsoAsp28] Exendin-4(1-39),

wherein the group-Lys6-NH2 may be bound to the C-terminus of the Exendin-4 derivative;

or an Exendin-4 derivative of the sequence

H-(Lys)6-des Pro36 [Asp28] Exendin-4(1-39)-Lys6-NH2,

des Asp28 Pro36, Pro37, Pro38Exendin-4(1-39)-NH2,

H-(Lys)6-des Pro36, Pro38 [Asp28] Exendin-4(1-39)-NH2,

H-Asn-(Glu)5des Pro36, Pro37, Pro38 [Asp28] Exendin-4(1-39)-NH2,

des Pro36, Pro37, Pro38 [Asp28] Exendin-4(1-39)-(Lys)6-NH2,

H-(Lys)6-des Pro36, Pro37, Pro38 [Asp28] Exendin-4(1-39)-(Lys)6-NH2,

H-Asn-(Glu)5-des Pro36, Pro37, Pro38 [Asp28] Exendin-4(1-39)-(Lys)6-NH2,

H-(Lys)6-des Pro36 [Trp(O2)25, Asp28] Exendin-4(1-39)-Lys6-NH2,

H-des Asp28 Pro36, Pro37, Pro38 [Trp(O2)25] Exendin-4(1-39)-NH2,

H-(Lys)6-des Pro36, Pro37, Pro38 [Trp(O2)25, Asp28] Exendin-4(1-39)-NH2,

H-Asn-(Glu)5-des Pro36, Pro37, Pro38 [Trp(O2)25, Asp28] Exendin-4(1-39)-NH2,

des Pro36, Pro37, Pro38 [Trp(O2)25, Asp28] Exendin-4(1-39)-(Lys)6-NH2,

H-(Lys)6-des Pro36, Pro37, Pro38 [Trp(O2)25, Asp28] Exendin-4(1-39)-(Lys)6-NH2,

H-Asn-(Glu)5-des Pro36, Pro37, Pro38 [Trp(O2)25, Asp28] Exendin-4(1-39)-(Lys)6-NH2,

H-(Lys)6-des Pro36 [Met(O)14, Asp28] Exendin-4(1-39)-Lys6-NH2,

des Met(O)14 Asp28 Pro36, Pro37, Pro38 Exendin-4(1-39)-NH2,

H-(Lys)6-desPro36, Pro37, Pro38 [Met(O)14, Asp28] Exendin-4(1-39)-NH2,

H-Asn-(Glu)5-des Pro36, Pro37, Pro38 [Met(O)14, Asp28] Exendin-4(1-39)-NH2,

des Pro36, Pro37, Pro38 [Met(O)14, Asp28] Exendin-4(1-39)-(Lys)6-NH2,

H-(Lys)6-des Pro36, Pro37, Pro38 [Met(O)14, Asp28] Exendin-4(1-39)-(Lys)6-NH2,

H-Asn-(Glu)5 des Pro36, Pro37, Pro38 [Met(O)14, Asp28] Exendin-4(1-39)-(Lys)6-NH2,

H-Lys6-des Pro36 [Met(0)14, Trp(02)25, Asp28] Exendin-4(1-39)-Lys6-NH2,

H-des Asp28 Pro36, Pro37, Pro38 [Met(O)14, Trp(O2)25] Exendin-4(1-39)-NH2,

H-(Lys)6-des Pro36, Pro37, Pro38 [Met(O)14, Asp28] Exendin-4(1-39)-NH2,

H-Asn-(Glu)5-des Pro36, Pro37, Pro38 [Met(O)14, Trp(O2)25, Asp28] Exendin-4(1-39)-NH2,

des Pro36, Pro37, Pro38 [Met(O)14, Trp(O2)25, Asp28] Exendin-4(1-39)-(Lys)6-NH2,

H-(Lys)6-des Pro36, Pro37, Pro38 [Met(O)14, Trp(O2)25, Asp28] Exendin-4(S1-39)-(Lys)6-NH2,

H-Asn-(Glu)5-des Pro36, Pro37, Pro38 [Met(O)14, Trp(O2)25, Asp28] Exendin-4(1-39)-(Lys)6-NH2;

or a pharmaceutically acceptable salt or solvate of any one of the afore-mentioned Exedin-4 derivative.

Hormones are for example hypophysis hormones or hypothalamus hormones or regulatory active peptides and their antagonists as listed in Rote Liste, ed. 2008, Chapter 50, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

A polysaccharide is for example a glucosaminoglycane, a hyaluronic acid, a heparin, a low molecular weight heparin or an ultra low molecular weight heparin or a derivative thereof, or a sulphated, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium.

Antibodies are globular plasma proteins (˜150 kDahttp://en.wikipedia.org/wiki/Dalton_%28unit%29) that are also known as immunoglobulins which share a basic structure. As they have sugar chains added to amino acid residues, they are glycoproteins. The basic functional unit of each antibody is an immunoglobulin (Ig) monomer (containing only one Ig unit); secreted antibodies can also be dimeric with two Ig units as with IgA, tetrameric with four Ig units like teleost fish IgM, or pentameric with five Ig units, like mammalian IgM.

The Ig monomer is a “Y”-shaped molecule that consists of four polypeptide chains; two identical heavy chains and two identical light chains connected by disulfide bonds between cysteine residues. Each heavy chain is about 440 amino acids long; each light chain is about 220 amino acids long. Heavy and light chains each contain intrachain disulfide bonds which stabilize their folding. Each chain is composed of structural domains called Ig domains. These domains contain about 70-110 amino acids and are classified into different categories (for example, variable or V, and constant or C) according to their size and function. They have a characteristic immunoglobulin fold in which two β sheets create a “sandwich” shape, held together by interactions between conserved cysteines and other charged amino acids.

There are five types of mammalian Ig heavy chain denoted by α, δ, ε, γ, and μ. The type of heavy chain present defines the isotype of antibody; these chains are found in IgA, IgD, IgE, IgG, and IgM antibodies, respectively.

Distinct heavy chains differ in size and composition; α and γ contain approximately 450 amino acids and δ approximately 500 amino acids, while μ and ε have approximately 550 amino acids. Each heavy chain has two regions, the constant region (C_{H}) and the variable region (V_{H}). In one species, the constant region is essentially identical in all antibodies of the same isotype, but differs in antibodies of different isotypes. Heavy chains γ, a and δ have a constant region composed of three tandem Ig domains, and a hinge region for added flexibility; heavy chains μ and ε have a constant region composed of four immunoglobulin domains. The variable region of the heavy chain differs in antibodies produced by different B cells, but is the same for all antibodies produced by a single B cell or B cell clone. The variable region of each heavy chain is approximately 110 amino acids long and is composed of a single Ig domain.

In mammals, there are two types of immunoglobulin light chain denoted byλ and κ. A light chain has two successive domains: one constant domain (CL) and one variable domain (VL). The approximate length of a light chain is 211 to 217 amino acids. Each antibody contains two light chains that are always identical; only one type of light chain, κ or λ, is present per antibody in mammals.

Although the general structure of all antibodies is very similar, the unique property of a given antibody is determined by the variable (V) regions, as detailed above. More specifically, variable loops, three each the light (VL) and three on the heavy (VH) chain, are responsible for binding to the antigen, i.e. for its antigen specificity. These loops are referred to as the Complementarity Determining Regions (CDRs). Because CDRs from both VH and VL domains contribute to the antigen-binding site, it is the combination of the heavy and the light chains, and not either alone, that determines the final antigen specificity.

An “antibody fragment” contains at least one antigen binding fragment as defined above, and exhibits essentially the same function and specificity as the complete antibody of which the fragment is derived from. Limited proteolytic digestion with papain cleaves the Ig prototype into three fragments. Two identical amino terminal fragments, each containing one entire L chain and about half an H chain, are the antigen binding fragments (Fab). The third fragment, similar in size but containing the carboxyl terminal half of both heavy chains with their interchain disulfide bond, is the crystalizable fragment (Fc). The Fc contains carbohydrates, complement-binding, and FcR-binding sites. Limited pepsin digestion yields a single F(ab′)2 fragment containing both Fab pieces and the hinge region, including the H-H interchain disulfide bond. F(ab′)2 is divalent for antigen binding. The disulfide bond of F(ab′)2 may be cleaved in order to obtain Fab′. Moreover, the variable regions of the heavy and light chains can be fused together to form a single chain variable fragment (scFv).

Pharmaceutically acceptable salts are for example acid addition salts and basic salts. Acid addition salts are e.g. HCl or HBr salts. Basic salts are e.g. salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1-C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology.

Pharmaceutically acceptable solvates are for example hydrates.

### BRIEF DESCRIPTION OF THE DRAWINGS

Further features, refinements and expediencies become apparent from the following description of the exemplary embodiments in connection with the figures.

FIG. 1 schematically shows a perspective view of an exemplary embodiment of a locking interface on a cartridge holder.

FIG. 2 schematically shows a side view of the locking interface of FIG. 1.

FIG. 3 schematically shows a perspective view of the cartridge holder of FIG. 1 locked to a main body of a drug delivery device.

Like elements, elements of the same kind and identically acting elements are provided with the same reference numerals in the figures.

### DETAILED DESCRIPTION

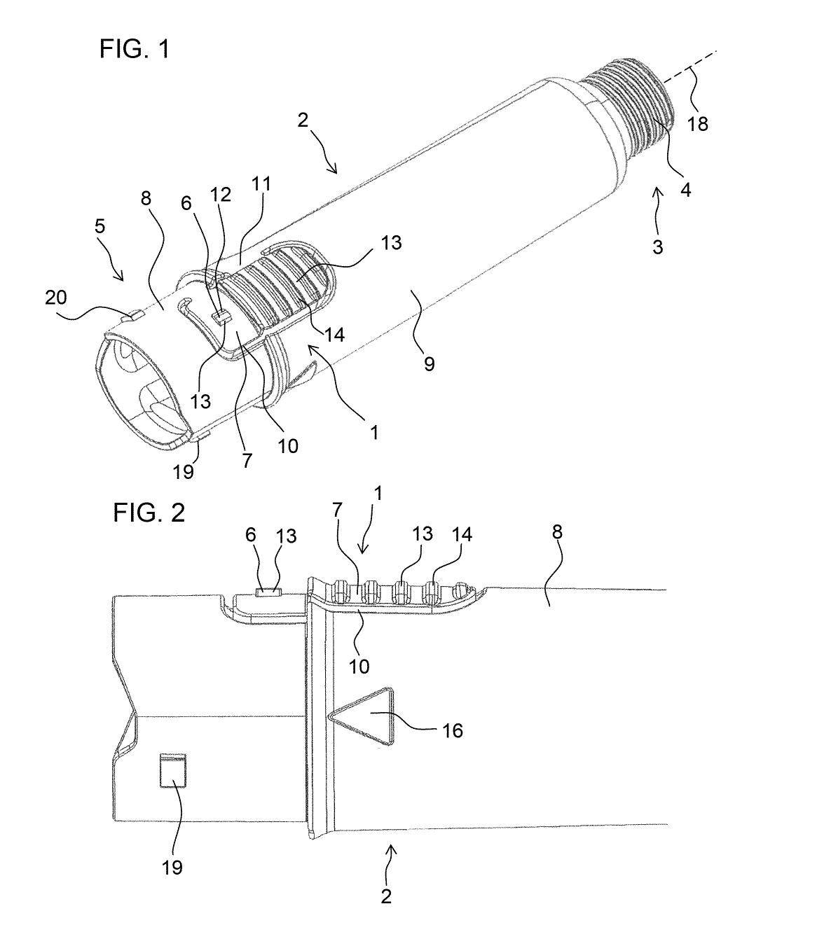

FIG. 1 shows a locking interface 1 located on a cartridge holder 2 for locking the cartridge holder 2 onto a main body of a drug delivery device. The cartridge holder 2 may comprise or made be made from a plastic material.

The depicted cartridge holder 2 may be suitable for an injection device, in particular a pen-type injection device. The cartridge holder 2 is configured for holding a cartridge containing a drug to be administered to a user. The cartridge holder 2 has a cylindrical shape and extends along a longitudinal axis 18.

At its distal end 3, the cartridge holder comprises a thread 4 for attaching a needle assembly. At its proximal end 5, the cartridge holder 2 comprises guide pins 19, 20 for attaching the cartridge holder 2 onto the main body of the device. At its distal end, the main body comprises one or more guide grooves (not shown here) for guiding the guide pins 19, 20. The guide grooves comprise an annular section or a helical section such that an attachment operation comprises a rotational movement, for example a screwing movement, of the cartridge holder 2 relative to the main body of the device, with the longitudinal axis 18 acting as the axis of rotation.

When the cartridge holder 2 is fully attached onto a distal end of the main body, it is locked in this position by the interaction of a locking member 6 of the locking interface 1 and a mating locking member of the main body of the device. Thereby, an inadvertent detachment of the cartridge holder 2 from the main body can be prevented.

The drug delivery device is configured as a reusable device such that the cartridge in the cartridge holder 2 can be replaced and the device can be reused with a new cartridge. For this aim, the cartridge holder can be unscrewed from the main part and thereby can be detached from the main part.

In the following, the function of the locking interface 1 is described in more detail.

The locking interface 1 comprises a locking member 6, which, in this embodiment, is shaped as a protruding lug. At its distal end, the main body of the device comprises a mating locking member, which may have the shape of a lug protruding inwardly from the main body of the device. Alternatively, the mating locking member may have the shape of a recess wherein the locking member may engage. At the end of the rotational movement of the cartridge holder relative to the main body, the mating protruding locking member may have to be pushed over the locking member 6 such that the cartridge holder 2 is fully attached and locked to the main part.

The locking member 6 is located on a support member 7 which is resiliently connected to a base 8. In this embodiment, the base 8 is configured as the housing 9 of the cartridge holder 2. The support member 7 is partially encircled by a cut-out 10 in the housing 9 and only connected to the housing 9 in an interface region 11 which acts as a hinge in the tilting movement. Thereby, when applying a force on the support member 7 in an inward direction of the cartridge holder 2, i.e., to the center of the cartridge holder 2, the support member 7 tilts relative to the housing 9.

When the cartridge holder 2 is screwed onto the main body, near the end of the screwing movement, an abutment of the locking member 6 on the mating locking member of the main part of the device occurs. Thereby, a force is exerted on a first interaction side 12 of the locking member 6. The first interaction side 12 is slanted such that when a mating locking member is pushed onto the first interaction side 12, a force directed to the center of the cartridge holder is exerted on the locking member 6. This results in a tilting motion of the support member 7 in an inward direction such that the mating locking member can be easily pushed over the locking member 6. Accordingly, by the design of the locking interface 1, a torque for locking the cartridge holder 2 onto the main body is reduced.

When the mating locking member has been fully pushed over the locking member 6 the support member 7 resiliently returns to its untilted position.

In the locked state, in order to unlock the cartridge holder 2, the mating locking member has to be pushed over the locking member 6 in the opposite direction such that the mating locking member interacts with a second interaction side 13 of the locking member 6. The second interaction side 13 is steeper than the first interaction side 12, such that by an interaction of the locking member 6 with the mating locking member only a small inwardly directed force is produced. Accordingly, the support member 7 does not tilt inwardly or only in a small amount. Accordingly, a high torque has to be applied in order to push the mating locking member over the locking member 6 in an unlocking direction. Thereby, an accidental detachment of the cartridge holder 2 from the main body is prevented.

In order to facilitate an intentional unlocking of the cartridge holder 2, the locking interface 1 comprises a user interface 11, which can be operated, i.e., depressed, in a locked state of the device. The user interface comprises tactile members 14 in the shape of ribs. By pressing onto the user interface 13, the user interface 13 and, thereby, the support member 7 tilts in an inward direction. Thereby, also the locking member 6 moves inwardly, such that the mating locking member can easily be pushed over the locking member 6 for unscrewing the cartridge holder 2 from the main body. Accordingly, the torque for unlocking the cartridge holder 2 is reduced by depressing the user interface 13 when unlocking the cartridge holder 2.

The user interface 13 is located at an offset relative to the locking member 6 in a direction towards the distal end 3. The center of the user interface 13 is located at the same distance from the hinge 11 as the locking member 6. Thereby, the amount of deflection of the user interface 13 and the locking member 6 are the same.

FIG. 2 shows a side view of the part of the cartridge holder of FIG. 1 where the locking interface 1 is located.

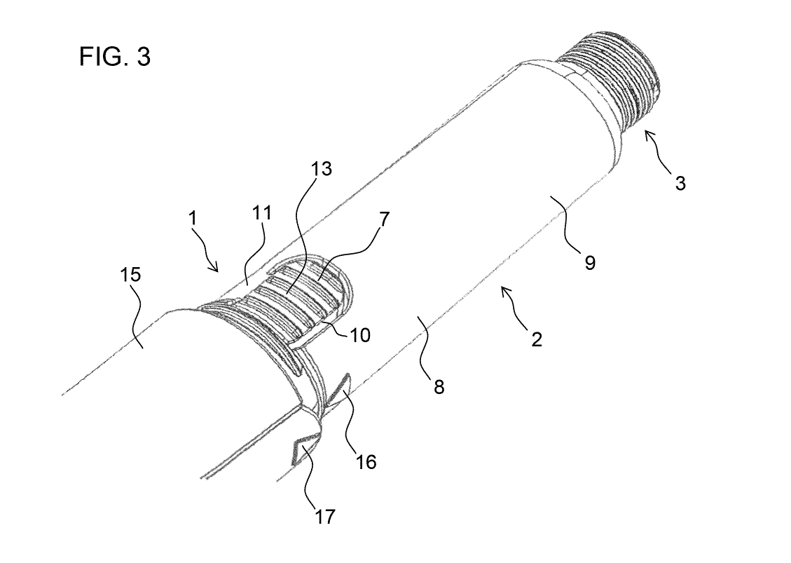

FIG. 3 shows the cartridge holder 2 fully mounted on the main body 15 of the device. Both the cartridge holder 2 and the main body 15 comprise a marking 16 resp. 17 in the shape of an arrow. Thereby, a user is informed that the cartridge holder 2 is fully screwed onto the main part and locked in this position.

1-15. (canceled)

16. A locking interface for locking a cartridge holder onto a main body of a drug delivery device, the locking interface comprising:
a locking member for locking the cartridge holder onto the main body,
a user interface operable by a user,
a support member, on which the locking member and the user interface are located, wherein the locking interface is configured such that a force needed for unlocking the cartridge holder from the main body is reducible by operating the user interface.

17. The locking interface of claim 16, comprising a base, wherein the support member is resiliently connected to the base.

18. The locking interface of claim 17, wherein the support member is resiliently connected to the base in a hinge region.

19. The locking interface of claim 17, wherein the resilient connection is facilitated by at least one cut-out in the base.

20. The locking interface of claim 19, wherein the cut-out encircles the support member except from the hinge region.

21. The locking interface of claim 17, configured such that by operating the user interface the support member deflects relative to the base.

22. The locking interface of claim 16, wherein the user interface comprises at least one tactile member.

23. The locking interface of claim 22, wherein the tactile member comprises at least one of a rib, a knob or a rippled surface.

24. The locking interface of claim 17, wherein the user interface and the locking member are located such on the support member that, when the user interface is operated, the locking member and the user interface deflect in the same amount relative to the base.

25. The locking interface of claim 17, wherein the base extends along a longitudinal axis and wherein the support member and the user interface are located in an offset along the longitudinal axis.

26. A cartridge holder attachable to a main body of a drug delivery device, wherein the cartridge holder comprises the locking interface of claim 16.

27. The cartridge holder of claim 26, configured to be attachable to the main body by a revolving movement of the cartridge holder or the main body.

28. A drug delivery device comprising a cartridge holder, a main body and a locking interface of claim 16, wherein the cartridge holder is attachable to the main body and lockable to the main body by the locking member.

29. The drug delivery device of claim 28 being configured as a reusable device.

30. The drug delivery device of claims 28, wherein the cartridge holder is attachable to the main body by a revolving movement of one of the cartridge holder and the main body, lockable to the main body by the locking member and configured such that a torque needed for unlocking the cartridge holder is reducible by operating the user interface.
